(19) United States
(12) Patent Application Publication
(10) Pub. No.: US 2016/0166218 A1
(43) Pub. Date: Jun. 16, 2016

(54) SYSTEM AND METHOD FOR EXPECTATION MAXIMIZATION RECONSTRUCTION FOR GAMMA EMISSION BREAST TOMOSYNTHESIS

(71) Applicant: UNIVERSITY OF VIRGINIA PATENT FOUNDATION, Charlottesville, VA (US)

(72) Inventors: Mark B. WILLIAMS, Earlysville, VA (US); Zongyi GONG, Charlottesville, VA (US)

(21) Appl. No.: 14/436,519

(22) PCT Filed: Oct. 18, 2013
(86) PCT No.: PCT/US2013/065731

§ 371 Date: Apr. 17, 2015

Related U.S. Application Data
(60) Provisional application No. 61/715,753, filed on Oct. 18, 2012.

(51) Int. Cl. A61B 6/00; A61B 6/02

(52) U.S. Cl. CPC A61B 6/4258; A61B 6/502; A61B 6/4417; A61B 6/5205; A61B 6/481; A61B 6/025

(57) ABSTRACT

A system and related methods for gamma emission breast tomosynthesis in which a set of two-dimensional images of a breast taken at different angular views are reconstructed into a three-dimensional map of the breast. The system applies an expectation maximization technique having integrated regularization, resolution recovery and attenuation correction to improve the clarity of the resulting three-dimensional map.

### CLAIM OF PRIORITY

This patent application claims the benefit of priority to U.S. Provisional Patent Application Ser. No. 61/715,753, filed on Oct. 18, 2012, which is hereby incorporated by reference herein in its entirety.

### TECHNICAL FIELD

This document pertains generally, but not by way of limitation, to systems for performing tomosynthesis of two-dimensional gamma-ray images and related methods of using.

### BACKGROUND

X-ray mammography is a common employed non-invasive breast cancer screening technique. X-ray mammograms involve compressing the breast to thin the tissue before administering an x-ray dose along a generally vertical axis to generate two-dimensional x-ray images of the breast. Typically, the patient is positioned in either a standing or seated position to generate top down or angled two-dimensional x-ray images of the breast. While x-ray mammograms are a widely accepted initial screening technique, the procedure has significant drawbacks. Specifically, radiodense tissue decreases mammographic sensitivity causing smaller lesions to be obscured by healthy tissue. As a result, cancerous tissue is often overlooked until the lesions have reach larger and more dangerous sizes. In addition, the limited two-dimensional views generated by x-ray mammograms are susceptible to false positives and often have false positive rates as high as 40%. As mammogram screening is supplemented with invasive tissue sampling testing such as biopsies, patients are often unnecessarily subjected to invasive, painful, and stressful testing as a result of false positives from mammograms.

Breast scintigraphy, called scintimammography, breast specific gamma imaging (“BSGI”) or molecular breast imaging (“MBI”) is a recently developed nuclear medicine procedure that can serve as a non-invasive adjunct imaging modality for x-ray mammography with the goal of enabling better depiction and characterization of smaller lesions in the breast. In this procedure, radiotracers, such as 99mTc-sestamibi, emitting gamma radiation that can be monitored with gamma-ray cameras are administered intravenously. The tracer accumulates preferentially in malignant cells, creating regions of higher tracer intensity at the locations of cancers within the breast. These regions of focal tracer uptake can be detected in the images of the emitted gamma rays. The higher energy gamma emission radiation of the radiotracers is less affected by variations in the radiodensity of the breast tissue thereby reducing the likelihood that small lesions will be obscured by radiodense healthy tissue. In addition, the use of small field of view (“FOV”) gamma cameras, which can be placed adjacent the chest wall and in contact with the breast, facilitates imaging of the tracer within the entire breast at higher spatial resolutions.

Currently, breast scintigraphy is a 2-dimensional imaging technique. The two-dimensional images produced provide no resolution in the third dimension. The lack of depth information prevents correction of the images to account for the impact of gamma ray-attenuation and depth dependent detector blurring. These physical factors result in poor detection for small or deeply seated lesions within the breast. Similarly, the two-dimensional images result in superposition of tracer distribution in breast structures throughout the breast, reducing image contrast and generating correlated background structural noise that can mask small lesions.

X-ray breast tomosynthesis (XBT) is a recently developed technique in which a series of two-dimensional x-ray images of the breast are obtained at a plurality of viewing angles over a limited angular range around the breast and digitally reconstructed to provide three-dimensional information regarding the breast structure. The three-dimensional information has been shown to improve the detection of small lesions and reduce false positives.

A tracer imaging technique developed by the inventors, gamma emission breast tomosynthesis (GEBT), applies a similar acquisition strategy as in XBT by exploring a limited angular range less than 180 degrees, typically 40 to 90 degrees, for acquisition of the projection views. In addition to providing three-dimensional images of the distribution of the tracer within the breasting, GEBT provides opportunities for corrections of the imaging degrading factors present in two-dimensional nuclear breast imaging, such as gamma ray attenuation and depth dependent camera blurring. However, the limited viewing angle results in an incomplete dataset for three-dimensional image reconstruction, which can result in image artifacts. As the limited views are frequently arranged in asymmetrical acquisition geometry (i.e. predominantly on one side of the breast), additional artifacts can be introduced.

### Overview

The present inventors have recognized, among other things, that a problem to be solved can include the difficulty of correcting image degrading factors inherent in nuclear breast imaging and correcting reconstruction artifacts inherent in tomosynthesis. In an example, the present subject matter can be provide a solution to this problem, such as by devising an expectation maximization (“EM”) reconstruction technique having integrated regularization, resolution recovery (“RR”) and attenuation correction (“AC”).

In an example, a plurality of two-dimensional projection images of radiotracer distribution within a breast are taken at a plurality of viewing angles within an angular range and digitally reconstructed into a three-dimensional image using an EM reconstruction technique. In at least one example, the EM reconstruction technique is a maximum likelihood expectation maximization (“MLEM”) technique. GEBT reconstruction reduces the superposition of tracer uptake in overlying breast structures and also improves the detection of small lesions while reducing false positives. In addition, the EM reconstruction technique reduces visual artifacts in the resulting three dimensional image generated from a set of images generated from a projection dataset that is angularly undersampled.

In an example, the EM reconstruction technique is regularized to prevent incorrect leakage of activity outside the breast region resulting from the undersampling of angular views. The regularization improves the quantitative nature of the depiction of the radiotracer concentration throughout the breast. In at least one example, the breast surface location is identified by a prior XBT analysis.

In an example, the EM reconstruction technique utilizes a volumetric inverse cone structure, a depth dependent camera blurring model and an attenuation factor applied to each iteration of the GEBT reconstruction. In at least one example, the volumetric inverse cone structure and depth dependent camera blurring factor are determined in part from the physical properties of the collimator associated with the gamma camera. In at least one example, the attenuation factors are determined in part from anatomical transmission data from the XBT scan to correct for attenuation by the breast of the gamma-rays. In at least one example, the AC relies on known attenuation properties of breast tissue at a given gamma emission energy.

In an example, resolution recovery (“RR”) is integrated into GEBT reconstruction in order to compensate for depth-dependent gamma camera blurring, improve overall spatial resolution, and reduce the spatial dependence of the resolution, and improve lesion contrast. The incorporation of AC and RR into the GEBT reconstruction removes attenuation artifacts and reduces the loss of lesion contrast that ordinarily occurs with increasing lesion depth in the breast. The improved spatial resolution provides improved three-dimensional localization of the lesions and can be used to guide subsequent procedures such as gamma-guided biopsy with improved accuracy.

The regularization, AC and RR also compensate for the limited angle acquisition geometry inherent in tomosynthesis to provide higher lesion contrast and signal to noise ratio (“SNR”) than is possible using planar scintimammography with an equal number of detected gamma events. Moreover, the effectiveness of the EM reconstruction techniques of the present subject matter in terms of spatial resolution, contrast, and SNR improves with increasing angular range of data acquisition.

In an example, the structural and functional breast gamma-ray images are obtained with a dual modality tomosynthesis (“DMT”) scanner that includes an x-ray component and a gamma-ray component for sequentially performing XBT and GEBT reconstructions, in which the EM technique for performing the GEBT reconstruction utilizes information found in the XBT reconstruction.

This overview is intended to provide an overview of the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic view depicting a representative acquisition geometry for two projection views.

FIG. 2A is a schematic view depicting a representative inverse cone for a parallel-hole collimation geometry.

FIG. 2B is a schematic view depicting a representative overlap of two aperture functions of a parallel-hole collimation geometry

FIG. 3 is a schematic view depicting the geometry for calculation of an attenuation factor, wherein a line integral for the attenuation along a ray axis is back-projected to off-axis voxels lying within a cone.

FIG. 4 is a schematic diagram of a dual-modality tomosynthesis scanner according to an example of the present subject matter.

FIG. 5A is a GEBT reconstruction of a simulated box phantom having a representative rectangular background region of interest providing scale for a square lesion record of interest for a lesion.

FIG. 5B is a GEBT reconstruction of a simulated box phantom having a representative rectangular region of interest for measuring an average background voxel value and a square region of interest for measuring the average lesion voxel value.

FIG. 6 is a graphical representation depicting theoretical predictions of depth-dependent detector blurring (analytical) and measurements of depth-dependent detector blurring from simulations and experiments.

FIG. 7A is a graphical representation plotting spatial resolutions in a reconstructed GEBT image as a function of lesion position in the z-direction with respect to AOR, wherein the angular range is 45 degrees.

FIG. 7B is a graphical representation plotting spatial resolutions in a reconstructed GEBT image as a function of lesion position in the z-direction with respect to AOR, wherein the angular range is 90 degrees.

FIG. 7C is a graphical representation plotting spatial resolutions in a reconstructed GEBT image as a function of lesion position in the z-direction with respect to AOR, wherein the angular range is 135 degrees.

FIG. 8A is a 2D projection of a simulated point source phantom, wherein the projection direction is along the z-axis perpendicular to the x-y plane.

FIG. 8B is a maximum intensity projection along the z-dimension of a GEBT reconstruction of the simulated point source phantom.

FIG. 8C is a maximum intensity projection along the y-dimension of a GEBT reconstruction of the simulated point source phantom.

FIG. 9A is a slice parallel to the x-z plane taken from a GEBT reconstruction of a simulated box phantom wherein the angular range is 45 degrees and neither regularization nor AC is applied.

FIG. 9B is a slice parallel to the x-z plane taken from a GEBT reconstruction of a simulated box phantom, wherein the angular range is 90 degrees and neither regularization nor AC is applied.

FIG. 9C is a slice parallel to the x-z plane taken from a GEBT reconstruction of a simulated box phantom, wherein the angular range is 135 degrees and neither regularization nor AC is applied.

FIG. 9D is a slice parallel to the x-z plane taken from a GEBT reconstruction of a simulated box phantom, wherein the angular range is 45 degrees and the GEBT reconstruction is regularized.

FIG. 9E is a slice parallel to the x-z plane taken from a GEBT reconstruction of a simulated box phantom, wherein the angular range is 90 degrees and the GEBT reconstruction is regularized.

FIG. 9F is a slice parallel to the x-z plane taken from a GEBT reconstruction of a simulated box phantom, wherein the angular range is 135 degrees and the GEBT reconstruction is regularized.

FIG. 9G is a slice parallel to the x-z plane taken from a GEBT reconstruction of a simulated box phantom, wherein the angular range is 45 degrees and the GEBT reconstruction is regularized and corrected for attenuation.

FIG. 9H is a slice parallel to the x-z plane taken from a GEBT reconstruction of a simulated box phantom, wherein the angular range is 90 degrees and the GEBT reconstruction is regularized and corrected for attenuation.

FIG. 9I is a slice parallel to the x-z plane taken from a GEBT reconstruction of a simulated box phantom, wherein the angular range is 135 degrees and the GEBT reconstruction is regularized and corrected for attenuation.

FIG. 10A is graphical representation of profiles through background regions of the reconstructions of FIGS. 9A, 9D and 9G.

FIG. 10B is graphical representation of profiles through background regions of the reconstructions of FIGS. 9B, 9E and 9H.

FIG. 10C is graphical representation of profiles through background regions of the reconstructions of FIGS. 9C, 9F and 9I.

FIG. 11 is a graphical representation of the normalized intensities of a plurality of lesions in a box phantom as a function of lesion depth.

FIG. 12A is a slice taken from a GEBT reconstruction of a simulated box phantom containing a plurality of lesions at different depths, wherein the slice is located at a depth of 2 cm and the GEBT reconstruction is regularized.

FIG. 12B is a slice taken from a GEBT reconstruction of a simulated box phantom containing a plurality of lesions at different depths, wherein the slice is located at a depth of 4 cm and the GEBT reconstruction is regularized.

FIG. 12C is a slice taken from a GEBT reconstruction of a simulated box phantom containing a plurality of lesions at different depths, wherein the slice is located at a depth of 6 cm and the GEBT reconstruction is regularized.

FIG. 12D is a slice taken from a GEBT reconstruction of a simulated box phantom containing a plurality of lesions at different depths, wherein the slice is located at a depth of 2 cm and the GEBT reconstruction is regularized and an attenuation correction is applied.

FIG. 12E is a slice taken from a GEBT reconstruction of a simulated box phantom containing a plurality of lesions at different depths, wherein the slice is located at a depth of 4 cm and the GEBT reconstruction is regularized and an attenuation correction is applied.

FIG. 12F is a slice taken from a GEBT reconstruction of a simulated box phantom containing a plurality of lesions at different depths, wherein the slice is located at a depth of 6 cm and the GEBT reconstruction is regularized and an attenuation correction is applied.

FIG. 13 is a graphical representation plotting lesion contrast versus lesion depth in GEBT images and in a planar image acquired under conditions of the same number of total detected gamma events.

FIG. 14 is a graphical representation plotting lesion SNR versus lesion depth in GEBT images and in a planar image acquired under conditions of the same number of total detected gamma events.

FIG. 15A is a slice taken from a GEBT reconstruction of a lesion in a compressed gelatin breast phantom, wherein the lesion and slice have depths of 1.6 cm and a circular orbit is used during acquisition.

FIG. 15B is a slice taken from a GEBT reconstruction of a lesion in a compressed gelatin breast phantom, wherein the lesion and slice have depths of 4.3 cm and a circular orbit is used during acquisition.

FIG. 15C is a slice taken from a GEBT reconstruction of a lesion in a compressed gelatin breast phantom, wherein the lesion and slice have depths of 1.6 cm and a spatial resolution maximization (“SRM”) orbit is used during acquisition.

FIG. 15D is a slice taken from a GEBT reconstruction of a lesion in a compressed gelatin breast phantom, wherein the lesion and slice have depths of 4.3 cm and a SRM orbit is used during acquisition.

FIG. 16 is a graphical representation plotting the normalized intensities of a plurality of lesions in a compressed gelatin breast phantom as a function of lesion depth, wherein a circular orbit is used during acquisition.

FIG. 17 is a graphical representation plotting the normalized intensities of a plurality of lesions in a compressed gelatin breast phantom as a function of lesion depth, wherein a SRM orbit is used during acquisition.

FIG. 18A is a graphical representation plotting lesion contrast as a function of lesion depth in GEBT images of a compressed gelatin breast phantom, wherein a circular orbit is used during acquisition.

FIG. 18B is a graphical representation plotting lesion contrast as a function of lesion depth in GEBT images of a compressed gelatin breast phantom, wherein a SRM orbit is used during acquisition.

FIG. 19A is a graphical representation plotting lesion SNR as a function of lesion depth in GEBT images of a compressed gelatin breast phantom, wherein a circular orbit is used during acquisition.

FIG. 19B is a graphical representation plotting lesion SNR as a function of lesion depth in GEBT images of a compressed gelatin breast phantom, wherein a SRM orbit is used during acquisition.

FIG. 20 is a schematic diagram of a gamma-ray scanner according to an example of the present subject matter.

FIG. 21 is a block diagram illustrating an example machine upon which any one or more of the techniques discussed herein may be performed.

### DETAILED DESCRIPTION

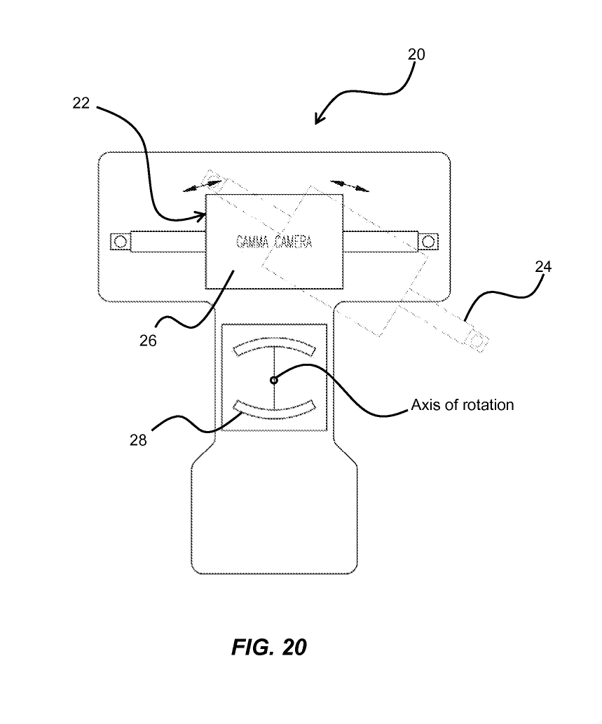

As depicted in FIG. 20, in an example of the present subject matter, a system 20 for collecting GEBT projection images includes a gamma-ray component 22 and a rotating gantry 24 for rotating the gamma-ray component 22 in a circular path about an axis of rotation (“AOR”). The gamma-ray component 22 includes a gamma-ray camera 26 having its detector surface oriented parallel to the AOR of the gantry 24. In an example, the gamma-ray component 36 comprises a low-energy parallel-hole collimator and a detector. In at least one example, the gamma camera 26 comprises a plurality of photomultiplier tubes arranged in a planar array and has about 13% FWHM energy resolution at 140 keV. In an example, the system 20 includes a support 28 positioned to support a breast at the AOR of the gantry 24.

In operation, the gantry 24 is rotated to position the central axis of gamma-ray camera 26 at a plurality of rotational angles within the angular range. In an example, the angular range is less than about 180 degrees. In at least one example, the angular range is less than about 90 degrees. In yet another example, the angular range is between about 30 degrees to about 90 degrees. In an example, the breast is positioned on the support 28 and compressed with a compression paddle 29 applying a compression force along an axis generally intersecting the AOR. The gamma-ray camera 26 is operable to measure gamma-ray radiation emitted by radiotracers within a breast positioned at the AOR. In an example, the radiotracer comprises ^{99m}Tc-sestamibi. The gamma-ray camera 26 creates a two-dimensional gamma-ray image of the breast at each corresponding angular view. In at least one example, the AOR-to-camera distance, as measured from the collimator surface, can be varied.

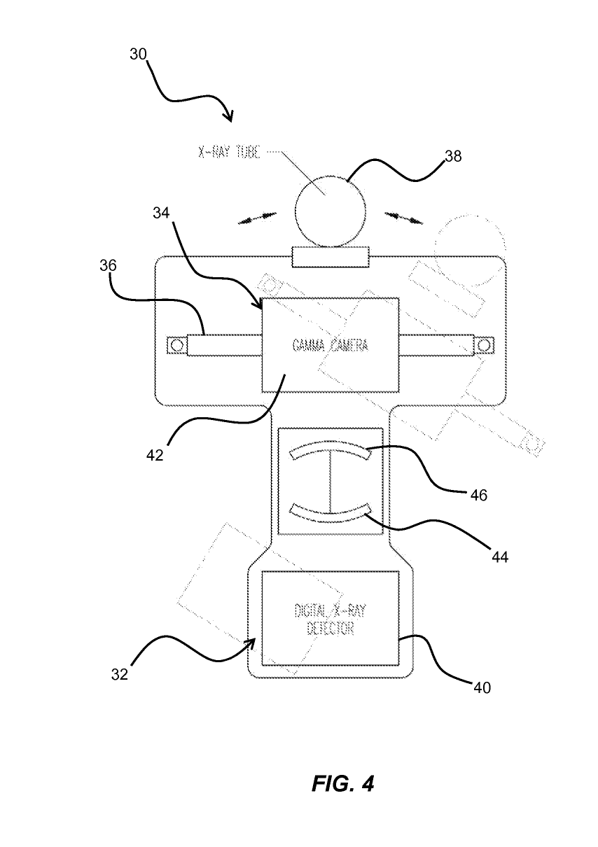

As depicted in FIG. 4, in an example of the present subject matter, a DMT scanner 30 for collecting structural and functional breast images includes an x-ray component 32, a gamma-ray component 34 and a rotating gantry 36 for rotating the x-ray component 32 and the gamma-ray component 34 in a circular path about an AOR. The x-ray component 32 includes an x-ray emitter 38 and an x-ray detector 40 defining a central ray intersecting the AOR of the gantry 36. The gamma-ray component 36 includes a gamma-ray camera 42 having a central surface normal oriented to intersect the AOR of the gantry 36. In an example, the gamma-ray component 36 comprises a low-energy parallel-hole collimator and a detector. In an example, the DMT scanner 30 also includes a support 44 positioned to support a breast at the AOR of the gantry 36.

In operation, the gantry 36 is rotated to position the central ray defined by the x-ray emitter 38 and x-ray detector 40 at a plurality of rotational angles within an angular range. In an example, the angular range is 180 degrees or less. At each rotational angle, the x-ray emitter 38 is operable to partially transmit an x-ray beam through a breast positioned on the support 44 at the intersection of the x-ray component central ray and the AOR. In an example, the breast is positioned on the support 44 and compressed with a compression paddle 46 applying a compression force along an axis generally intersecting the AOR. The x-ray detector 40 receives the transmitted x-ray beam to create a two-dimensional x-ray image of the breast at the corresponding angular view. Similarly, the gantry 36 is rotated to position the central axis of gamma-ray camera 42 at a plurality of rotational angles within the angular range. The gamma-ray camera 42 is operable to measure gamma-ray radiation emitted by radiotracers within a breast positioned near the gantry AOR. The gamma-ray camera 42 creates a two-dimensional gamma-ray image of the breast at each corresponding angular view. In at least one example, the AOR-to-camera distance, as measured from the collimator surface, can be varied.

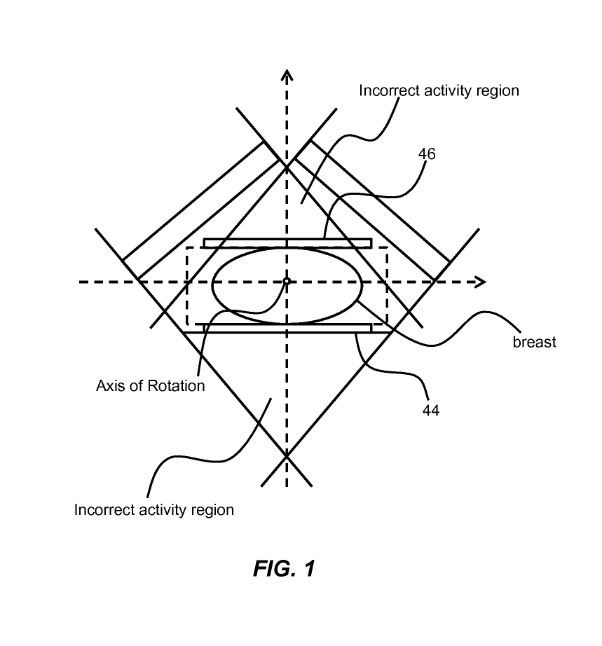

The limited set of angular views provides insufficient information to prevent reconstructed activity from being erroneously attributed to regions outside the breast in tomosynthesis reconstruction. Specifically, the reconstructed activity is erroneously attributed to regions outside the breast in an undersampled direction as illustrated in FIG. 1. In at least one example, the EM reconstruction frame can comprise a maximum likelihood expectation maximization (ML-EM), maximum a posterior expectation maximization (MAP-EM) or their ordered subset (OS) equivalences, ML-OS-EM or MAP-OS-EM. In an example, the EM technique is regularized based on anatomical transmission data from the XBT reconstruction to limit the activity distribution to the defined breast region. In another example, the breast region could be physically measured as defined by the borders of a breast support and a compression paddle. In an example, the regularized ML-EM update executed at the (n+1)^{th} iteration is expressed as:

$${f}_{j}^{0} = \begin{matrix}0 for j ∈ Ω \\ 1 for j ∉ Ω\end{matrix}$$

$${f}_{j}^{n + 1} = \frac{{f}_{j}^{n}}{{\sum }_{i} {a}_{ij} {b}_{ij}} {\sum }_{i} {a}_{ij} {b}_{ij} \frac{{p}_{i}}{{\sum }_{k} {a}_{ik} {b}_{ik} {f}_{k}^{n}}$$

fⱼ is the radioactivity to be reconstructed for voxel j, pᵢ is the number of detected counts at the detector bin i, aᵢⱼ is the depth dependent camera blurring, bᵢₙ is the attenuation factor, and Ω denotes the breast region between the support and compression paddle as determined from the XBT reconstruction. The matrix f is initialized by setting the value of voxels outside the breast region Ω to zero. The regularized EM equation assures that voxels not lying in breast region Ω maintain a zero value in subsequent iterations. In an example, the breast region Ω is defined by other mechanical, optical or fiducial methods than XBT reconstruction.

In an example, the depth dependent camera blurring aᵢⱼ is expressed as a normalized Gaussian function whose shape is primarily determined by the parameters of the parallel-hole collimator, the intrinsic resolution of the detector and the voxel j's location relative to the detector bin i:

$${a}_{ij} = \frac{1}{2 π {σ}_{ij}^{2}} exp \{ - \frac{{( {r}_{j} - {q}_{j} )}^{2}}{2 {σ}_{ij}^{2}} \}$$

$$\sqrt[2]{2 ln 2 {σ}_{ij }} = {R}_{s} = \sqrt{{R}_{c}^{2} + {R}_{i}^{2}} = \sqrt{{( D \frac{Z}{L} )}^{2} + {R}_{i}^{2}}$$

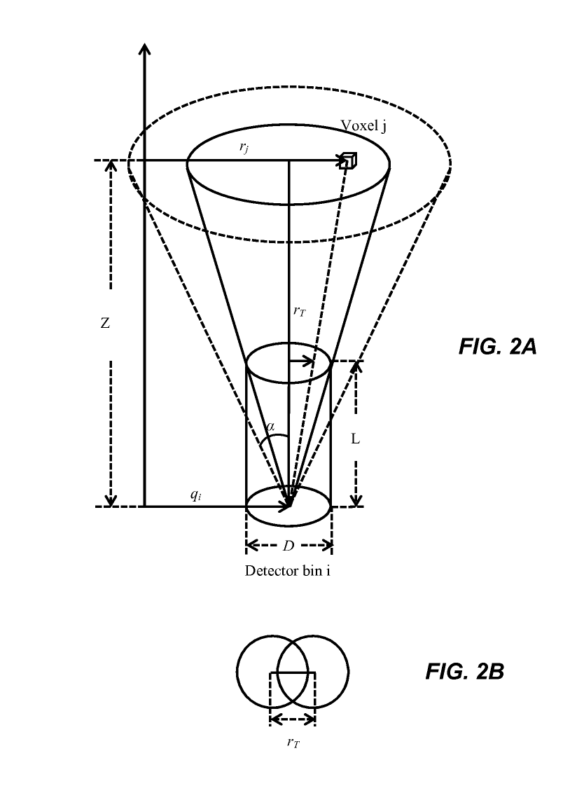

As depicted in FIG. 2A, rⱼ denotes the transverse locations of the voxel and qᵢ denotes the transverse locations of the detector bin measured in the detector plane. Z denotes the perpendicular distance of the j^{th} voxel above the detector surface. D is the diameter of the collimator holes and L is the length of the collimator holes. R_{c} is the collimator resolution and Rᵢ is the intrinsic resolution of the detector. Rₛ is the camera resolution defined as the full width at half maximum (“FWHM”) of the Gaussian blurring function.

In at least one example, blurring caused by the detector bin is negligible compared to the blurring caused by the collimator. In this configuration, the dimensions of a volumetric inverse cone projector-backprojector is determined by considering only voxels having sufficiently small transverse offsets relative to the location of the detector bin of interest such that the voxels can contribute counts as determined by the physical parameters of the parallel hole collimator. In one example, the geometric response function of the collimator is evaluated as the overlap of two aperture functions, offset by a distance r_{T} as depicted in FIG. 2B, referenced to the collimator hole entrance:

$${r}_{T} = \frac{L}{Z} ( {r}_{j} - {q}_{i} )$$

r_{T} is the distance between the source voxel and the axis of the collimator hole. r_{T}=The circular region with diameter D at height L corresponds to points positioned on the surface of the cone of acceptance of the collimator, which has a cone angle of:

$$α = arctan ( \frac{D}{L} )$$

In at least one example, the geometric response function of the collimator is evaluated as an autocorrelation of the front aperture. In this configuration, the cone diameter is twice the FWHM of the Gaussian blurring function if the intrinsic resolution term of the Gaussian blurring function is neglected.

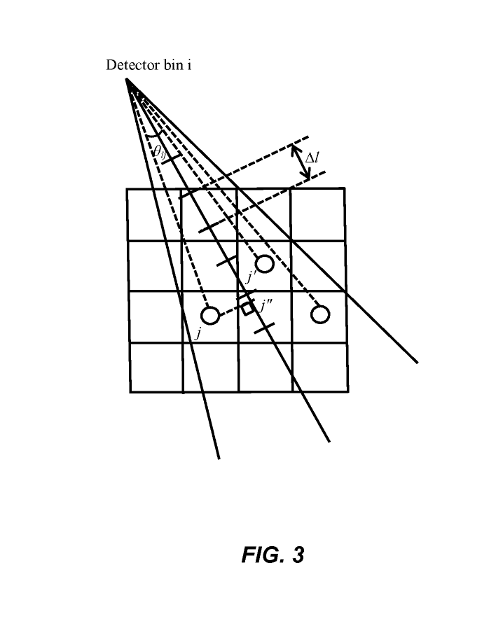

In at least one example, the attenuation factor bᵢⱼ is assumed to remain relatively constant over distances comparable to the cone diameter for all voxels within the inverse cone in order to avoid calculating the attenuation factors as individual line integrals for each voxel explicitly. Accordingly, the attenuation factors for individual voxels within the cone are estimated from the attenuation factors calculated only along the cone axis as depicted in FIG. 3. In this configuration, the attenuation factor bᵢⱼ for voxel j is expressed as:

$${b}_{ij} = exp \{ - \frac{1}{cos {θ}_{ij}} {\sum }_{k = 0}^{{j}^{′}} {μ}_{k} Δ l \}$$

θij is the angular distance of voxel j away from the cone axis centered at detector bin i. Δl is the length of a line segment along the ray axis which for simplicity is set to be equal to the voxel side length. μₖ is the linear attenuation coefficient at the midpoint of each line segment and calculated using trilinear interpolation from the nearest eight voxels. j′ is the index of the segment midpoint closest to the projection point j″ of the center of voxel j onto the cone axis. In an example, the summation of μₖ Δl is performed from the detector bin to segment j′.

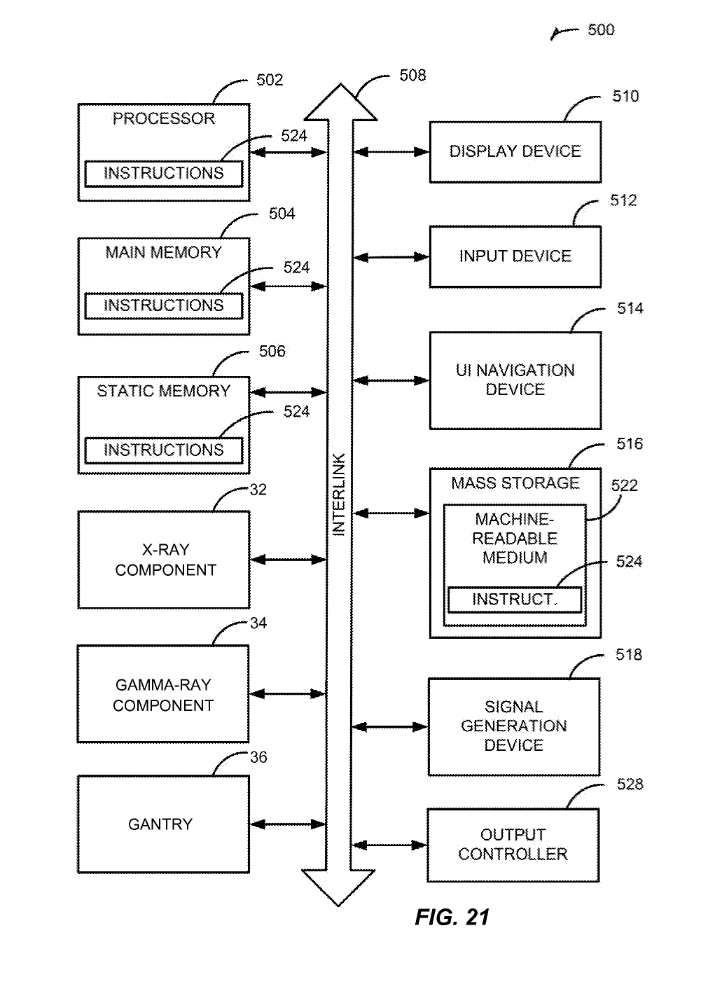

FIG. 21 is a block diagram illustrating an example machine 500 upon which any one or more of the techniques (e.g., methodologies) discussed herein may be performed. In alternative embodiments, the machine 500 may operate as a standalone device or may be connected (e.g., networked) to other machines. In a networked deployment, the machine 500 may operate in the capacity of a server machine, a client machine, or both in server-client network environments. In an example, the machine 500 may act as a peer machine in peer-to-peer (P2P) (or other distributed) network environments. The machine 500 may be a personal computer (PC), a tablet PC, a Personal Digital Assistant (PDA), a mobile telephone, a web appliance, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside (1) on a non-transitory machine-readable medium or (2) in a transmission signal. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software; the general-purpose hardware processor may be configured as respective different modules at different times. Software may accordingly configure a hardware processor, for example, to constitute a particular module at one instance of time and to constitute a different module at a different instance of time.

Machine (e.g., computer system) 500 may include a hardware processor 502 (e.g., a processing unit, a graphics processing unit (GPU), a hardware processor core, or any combination thereof), a main memory 504, and a static memory 506, some or all of which may communicate with each other via an interlink 508 (e.g., a bus, link, interconnect, or the like). The machine 500 may further include a display device 510, an input device 512 (e.g., a keyboard), and a user interface (UI) navigation device 514 (e.g., a mouse). In an example, the display device 510, input device 512, and UI navigation device 514 may be a touch screen display. The machine 500 may additionally include a mass storage (e.g., drive unit) 516, a signal generation device 518 (e.g., a speaker) and a network interface device 520. The machine 500 may additionally be operably linked to gantry 24, 36; the gamma-ray component 22, 34; and the x-ray component 32 for controlling operation thereof.

The mass storage 516 may include a machine-readable medium 522 on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the mass storage 516 may constitute machine readable media.

While the machine-readable medium 522 is illustrated as a single medium, the term “machine readable medium” may include a single medium or multiple media (e.g., a centralized or distributed database, and/or associated caches and servers) that configured to store the one or more instructions 524.

The term “machine-readable medium” may include any tangible medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine-readable medium examples may include solid-state memories, and optical and magnetic media used for storing data structures or instructions; and all such memory devices and storage media (whether discrete or integrated with other functionality, for example as cache memory) represent non-transitory media. Specific examples of machine-readable media may include: non-volatile memory, such as semiconductor memory devices (e.g., Electrically Programmable Read-Only Memory (EPROM), Dynamic Random Access Memory (DRAM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

### VARIOUS NOTES & EXAMPLES

### Example and Experimental Results

Practice of an example (or examples) of the present subject matter will be still more fully understood from the following examples and experimental results, which are presented herein for illustration only and should not be construed as limiting the invention in any way.

In the following experimental results, the performance of the EM reconstruction technique was evaluated in terms of resolution recovery (“RR”), artifact reduction, and attenuation correction (“AC”). Similarly, the FWHM of the position-dependent system point spread function (“PSF”), background uniformity, the depth dependence of the lesion voxel values and the lesion contrast and SNR were also evaluated.

### Point Source Simulation Setup

In an example, a 5-row by 5-column array of 25 spherical point sources are arranged in a planar array in the air was simulated, wherein the spherical point sources of a given row are positioned in the same plane in the z-direction and are separated by 3 cm from each adjacent spherical point source in the x and y directions. Each spherical point source is 0.02 mm in diameter.

The gamma-ray camera 42 is tested at z-coordinates of the rows of point sources were: −4 cm, −2 cm, 0 cm, +2 cm and +4 cm with respect to the AOR (located at z=0). In an example, the circular path of the gamma camera had a radius of 9.85 cm measured from the collimator surface to the AOR. Nine angular views were acquired over an angular span of 135 degrees. In an example, an angular view in which the collimator surface was placed 4.1 cm away from the AOR or almost touching the first row of spherical point sources was obtained to simulate planar scintimammography.

### Phantom Box Simulation Setup

In an example, a gelatin phantom breast was modeled as a rectangular parallelepiped 15 cm long by 15 cm wide by 8 cm deep. The phantom breasts are approximately 7.7 cm in thickness. In an example, the compressed breast thickness is between about 6% and about 28% greater during a DMT analysis than the compressed breast thickness for a conventional mammogram.

The evaluated phantom box contained three simulated lesions having the same x-coordinates, evenly spaced y-coordinates and z-coordinates that are positioned 2 cm, 4 cm and 6 cm from the top surface of the phantom box. Each lesion was spherical and had 15 mm diameters. The phantom lesions were filled with a ^{99m}Tc-water solution with 10:1 target to background activity concentration ratio (T:B).

In an example, the circular path of the camera had a radius of 9.85 cm measured from the collimator surface to the AOR. Nine angular views were acquired over angular spans of 45, 90 and 135 degrees to test the robustness of the EM reconstruction technique and assess the relative impacts of correction techniques with changing angular range. The acquisition time per frame was adjusted to result in a projection view count density of 130 cts/cm2 when the collimator surface was parallel to the 15 cm×15 cm surface of the phantom to simulate actual conditions. The projection view count density of 130 cts/cm2 is consistent with nine projection views using a 10 minute total scan time with a total scan count density of approximately 1150 cts/cm². For comparison of GEBT with planar breast scintimammography, a single projection resulting from the same number of detected photons as in GEBT was simulated with the collimator surface in contact with the top (15 cm×15 cm) surface of the phantom.

### Phantom Breast Simulation Setup

In an example, the intrinsic spatial resolution of a gamma-ray camera was determined by placing a 0.8 mm diameter ^{99m}Tc capillary source at a small angle with respect to the crystal array matrix and translating the capillary source in 1 cm steps between 0 and 12 cm from the surface of the collimator. In this configuration, row-by-row profiles for each capillary-to-collimator separation and through the line source images were fitted to Gaussian functions and their respective FWHMs were recorded.

In an example, gelatin phantom breasts having a volume of 840 ml and including two thin-walled spherical lesions with 15 mm interior diameters were imaged. The volume of the gelatin phantom was determined as the product of an average breast thickness of 7.7 cm and an image-based assessment of the average projected breast area. The lesion-to-background activity concentration ratio was 10:1. In an example, the gelatin phantom breast was compressed to a thickness of 7.7 cm to position the lesions about 1.6 cm and 4.3 cm bellow the top surface of the phantom breast.

Gamma-ray images for GEBT reconstruction were acquired by collecting twenty-five equally spaced projection images at a number of different angular ranges up to 135 degrees. A 9-view subset was selected from the twenty five images and grouped to form 3 scans of the same acquisition time and different angular spans (45 degrees, 90 degrees and 135 degrees). The count density per view was 130 cts/cm². Both circular (10 cm radius) and spatial resolution-maximized (SRM) orbits in which the gamma-ray camera was positioned as close as possible to the phantom in each view were tested.

For comparison of GEBT with planar breast scintimammography, a single projection was obtained with the collimator surface in contact with the 2 mm thick top (15 cm×15 cm surface area) of the acrylic box. The total number of detected events was adjusted to approximate that of complete GEBT scans. For the purpose of attenuation correction during reconstruction, the linear attenuation coefficient of the gelatin was measured using narrow beam transmission geometry and a collimated ^{99m}Tc flood source.

### Reconstruction and Image Analysis

For each simulation, the geometrical collimator response and projector-backprojector according to an example described or inherently present herein was applied to the GEBT reconstruction.

The uniform linear attenuation coefficient μₖ for the simulated water phantom data was assumed to be 0.150 cm⁻¹, which corresponds to the linear attenuation coefficient of water at 140 keV. The uniform linear attenuation coefficient μₖ for the gelatin phantom data was assumed to be 0.149 cm⁻¹. The reconstructions for the box phantom simulation were performed without the mask as the point sources are in air. Instead, the reconstructions for the box phantom simulation were performed: without AC and without the breast region regularization; without AC and with the regularization; and with both AC and the regularization. For the gelatin phantom data, reconstructions were performed with either AC or no AC but always with regularization.

Before reconstruction, the simulated projection data were rebinned from 128×128 into 64×64 matrix size and the experimental projection data were rebinned from 150×110 into 75×55 matrix size. The reconstructed volume matrix was 80×80×80 for the simulations and 94×94×69 for the experimental data, with 2.24 mm isotropic voxel size in both cases. Nine iterations on three ordered subsets of a total of nine views were conducted because the rate of improvement of lesion contrast in the reconstructed images with further iteration decrease after the first iterations.

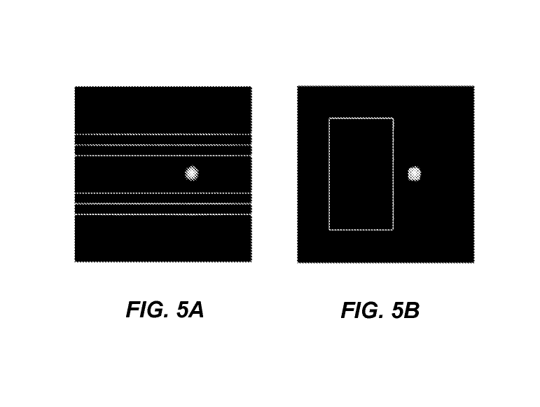

For the simulated point source data, the centers of twenty five point sources in the reconstructed volume were found. A plurality of one dimensional profiles through each point source in the x, y and z directions were drawn and fitted with Gaussian functions. The FWHMs of the Gaussians for the five sources located at the same z coordinate are averaged and reported as the three-dimensional resolutions at that z location. The spatial resolutions of the planar scintimammography images of the same phantom in the x and y directions were also measured. For the simulated box phantom data, the background uniformity of the reconstructed volumes was assessed by first summing the four slices closest to the middle plane (the plane containing the x and y axes) then extracting profiles on either side of the 4 cm deep lesion, as illustrated in FIG. 5A, and finally averaging the profiles. Lesion intensity and background intensity were measured in single slices located at each of the lesion centers. The background intensity was defined in the simulations as the average background pixel value in a 50×25 pixel region of interest (ROI) near the lesion and lesion intensity was defined as the average pixel value in a 4×4 pixel ROI centered on the lesion, as illustrated in FIG. 5B. In an example, lesion contrast and signal to noise ratio (“SNR”) were expressed as:

$$Contrast = \frac{{N}_{L} - {N}_{B}}{{N}_{B}}$$

$$SNR = \frac{{N}_{L} - {N}_{B}}{{σ}_{B}}$$

N_{B} is the average pixel value of the background ROI. σ_{B} is the standard deviation of the background ROI. N_{L} is the average pixel value in the lesion ROI. In the gelatin phantom image simulation, lesion contrast was measured in a similar way as in the simulated data, but with the size of the background ROI adjusted to 20×30 pixels.

### Point Source Simulation Results

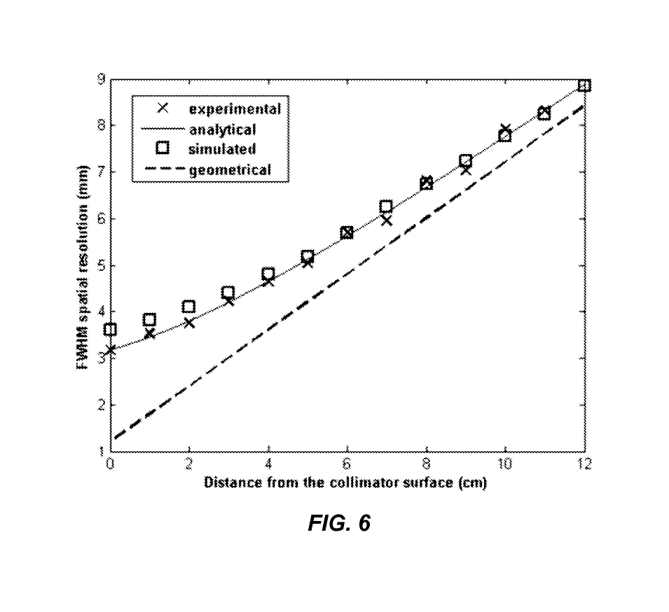

In the point source simulation, the depth dependent camera blurring aᵢⱼ was predicted for the spherical point source using the normalized Gaussian function and compared to experimental measurement as depicted in FIG. 6. The FWHMs of the imaged line source are measured at a series of capillary-to-collimator distances to provide an experimental trend line. The FWHMs of the simulated point sources are measured and plotted to provide a simulated trend line. Similarly, the collimator resolution R_{c} was plotted to provide a geometrical trend line and the calculated camera resolution Rₛ was plotted to provide an analytical trend line. The plotting of the predicted depth dependent camera blurring aᵢⱼ revealed that the planar resolution in the x and y dimensions decreases rapidly with increasing source distance.

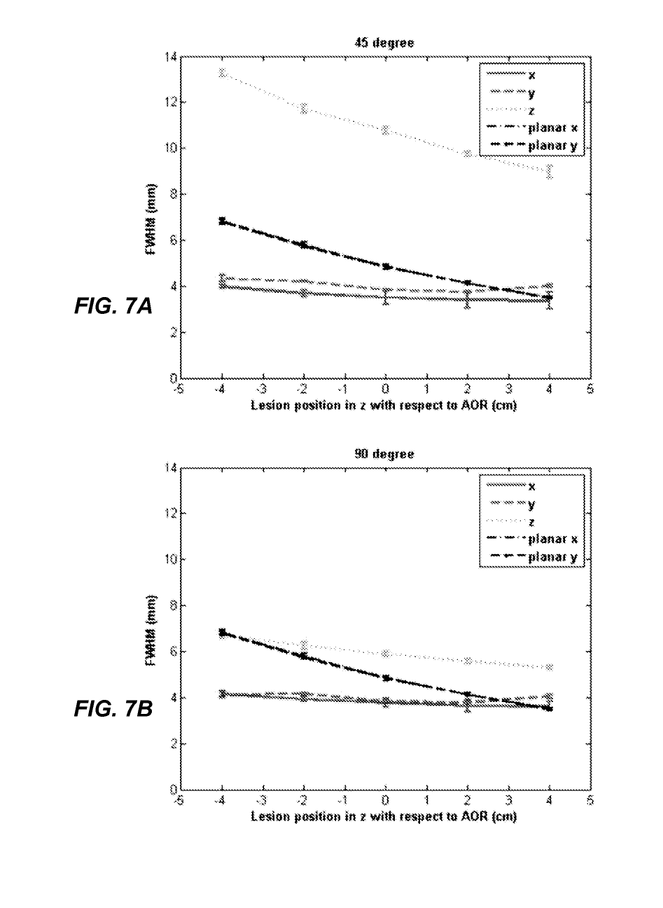

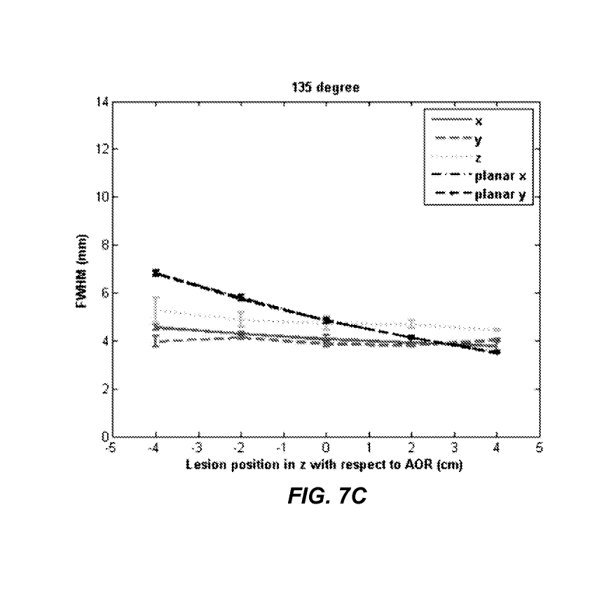

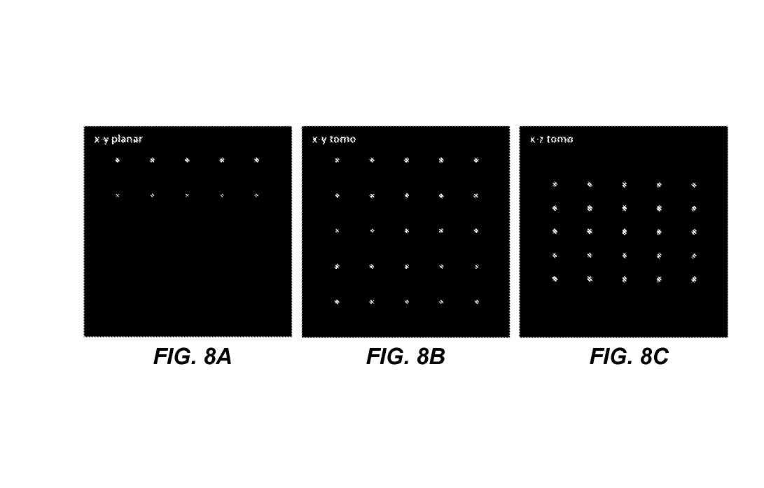

The x and y dimension resolutions in GEBT are generally independent of source location with minimal dependence on acquisition angular range. While GEBT also provides z-dimension resolution, the z resolution substantially depends on angular range and improves as the angular span increases as depicted in FIGS. 7A-7C. As depicted in FIG. 7A, at 45 degree angular range the z-resolution is degraded as the source-to-collimator distance at the 0 degree viewing angle increases such as when the source has a negative z coordinate. Similarly, as depicted in FIG. 7B the z-resolution with changing source-to-collimator separation is also degraded with increasing source-to-collimator separation for a 90 degree angular range, however to a lesser degree than with 45 degree angular range. FIG. 7C shows improved and nearly constant z-dimension spatial resolution at all source-to-collimator separations. As depicted in FIGS. 8A-8C, the 135 degree angular range results in near-isotropic and spatially uniform resolutions, wherein the FWHM of the sources is 4.12±0.31 mm in the x direction, 3.95±0.15 mm in the y direction and 4.79±0.39 mm in the z direction in an example.

### Phantom Breast Simulation Results

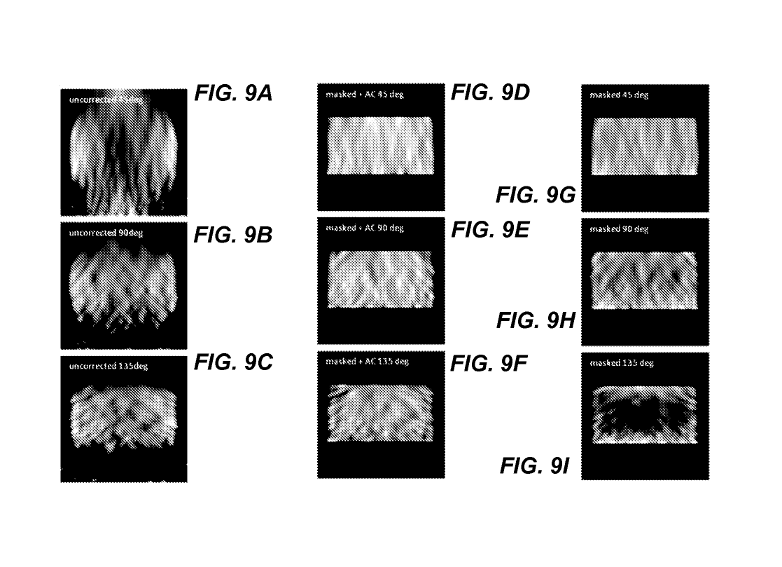

In an example, the GEBT of the phantom breast was evaluated without either regularization or AC, with regularization only and with both regularization and AC. As illustrated in FIGS. 9A-9I, if no correction is applied, leakage of activity from the upper and lower phantom surfaces will occur due to the incomplete angular sampling. The severity of the leakage decreases with increasing angular range. The application of regularization via masking according to an example removes the activity leakage as depicted in FIG. 9D-9F. Similarly, the application of AC according to an example increases the uniformity of the background intensity and lessens streak artifacts resulting from undersampling.

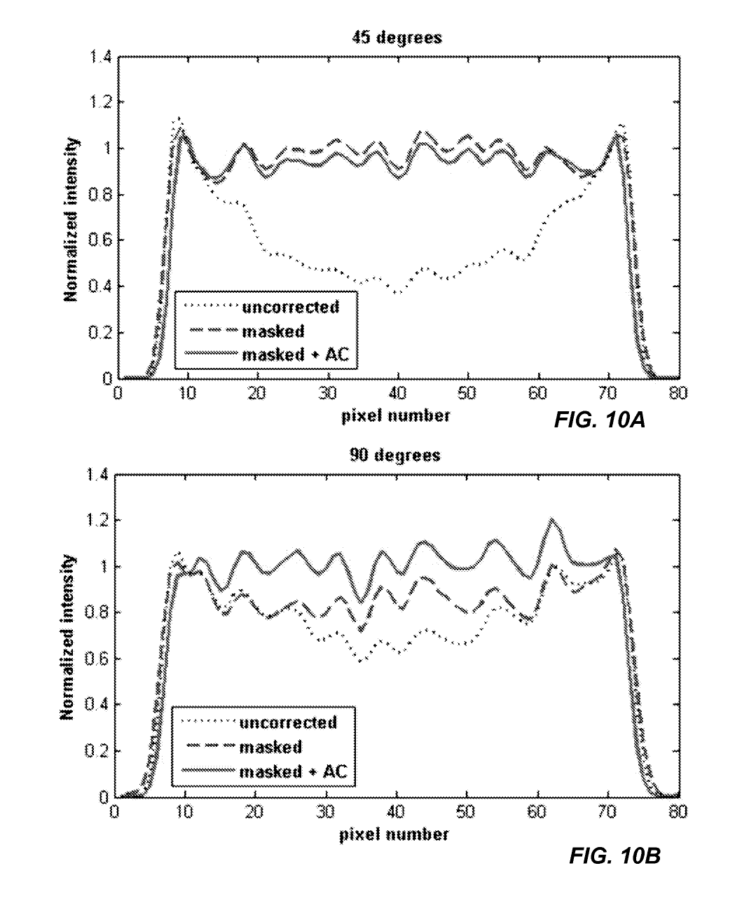

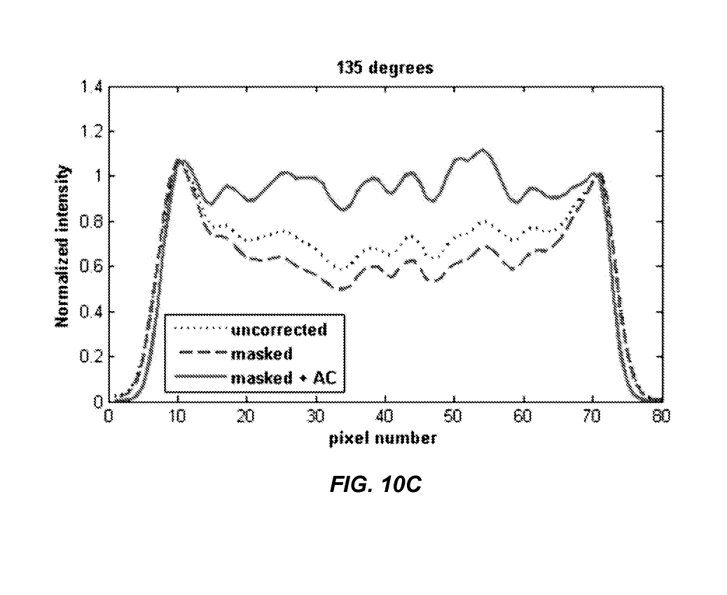

Similarly, as illustrated in FIG. 10A-10C, x-y plane slices of the reconstructions depicted in FIGS. 9A-9I produce cupping artifacts when regularization and AC are not applied. The cupping artifacts result from underestimation of the activity concentration in the breast region because of activity leaking from the upper and lower phantom surfaces due to undersampling and uncorrected attenuation. As depicted in FIG. 10A, applying regularization according to an example is more effective for removing cupping artifacts than AC for smaller angular ranges. Similarly, as depicted in FIG. 10C, applying AC according to an example is more effective than regularization for removing the cupping artifacts for larger angular ranges.

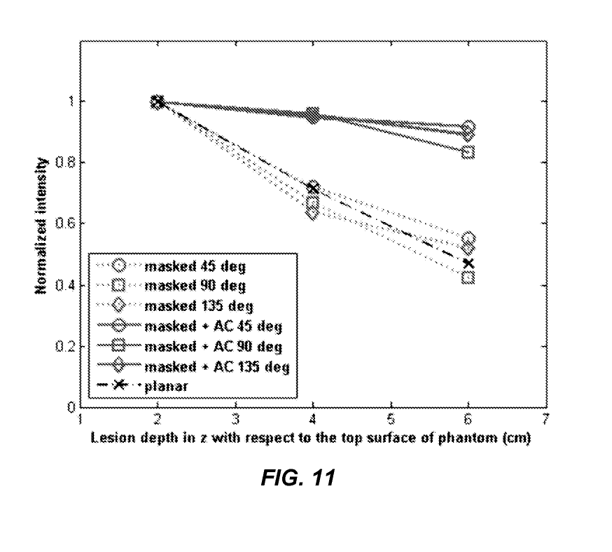

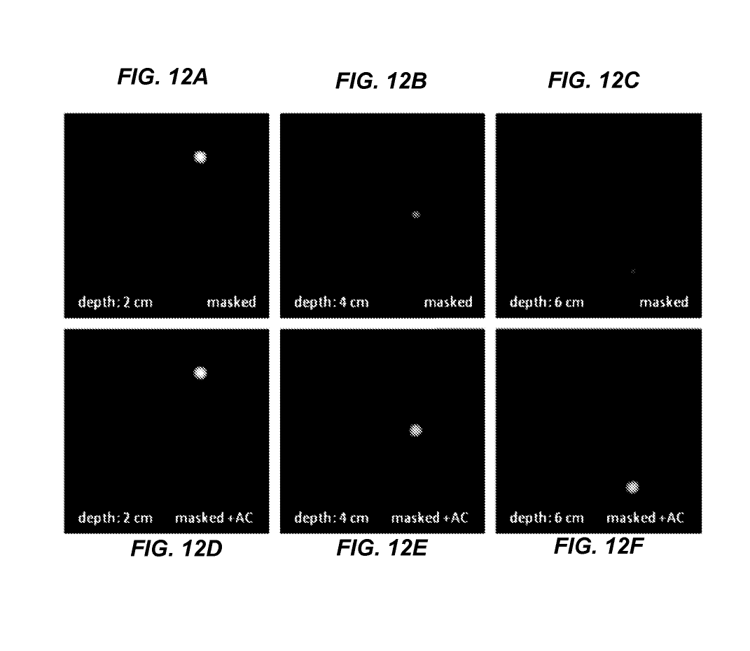

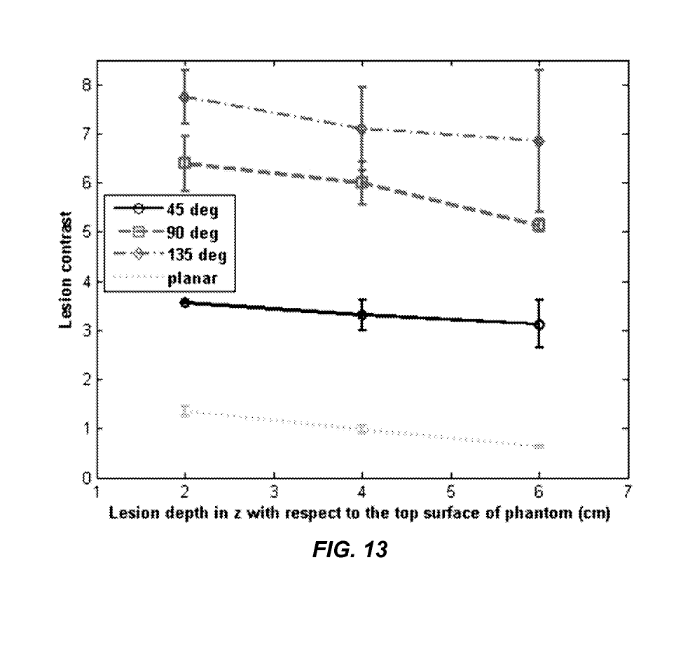

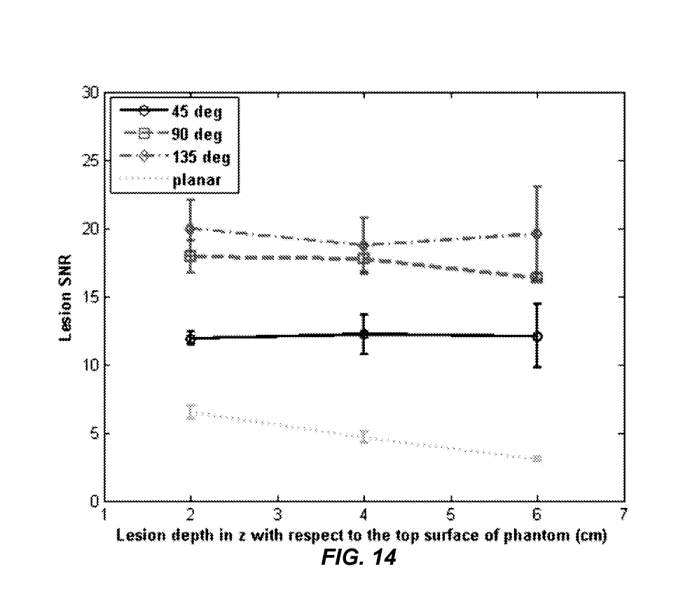

The change in the intensity of lesions with changing lesion depths was evaluated with regularization only and with both regularization and AC. As depicted in FIG. 11, the application of AC according to an example of the present subject matter reduces the decrease in lesion intensity with increasing lesion depth. In at least one example, the application of AC reduced the intensity difference between a lesion at a 2 cm depth and a lesion at a 4 cm depth from about 30% to less than 6%. In at least one example, the application of AC reduced the intensity difference between a lesion at a 4 cm depth and a lesion at a 6 cm depth from about 50% to less than 12%. Similarly, as depicted in FIG. 12A-12F, the visual intensity of lesions at lower depths is improved with the application of AC according to an example of the present subject matter, thereby demonstrating the effectiveness of AC in providing uniform lesion intensity. As depicted in FIG. 13, the lesion contrast was improved and the effect of lesion depth on lesion contract was reduced with the application of regularization and AC according to an example. In an example, the lesion contrast was reduced by 12% for a 45 degree angular range, 19% for a 90 degree angular range and 13% for a 135 degree angular range. Similarly, as depicted in FIG. 14, the lesion SNR was improved and the variation of the SNR due to increasing lesion depth was also reduced with the application of regularization and AC according to an example. In an example, the lesion SNR was reduced by less than 10% for the 45, 90 and 135 degree angular ranges.

### Spatial Resolution Simulation Setup

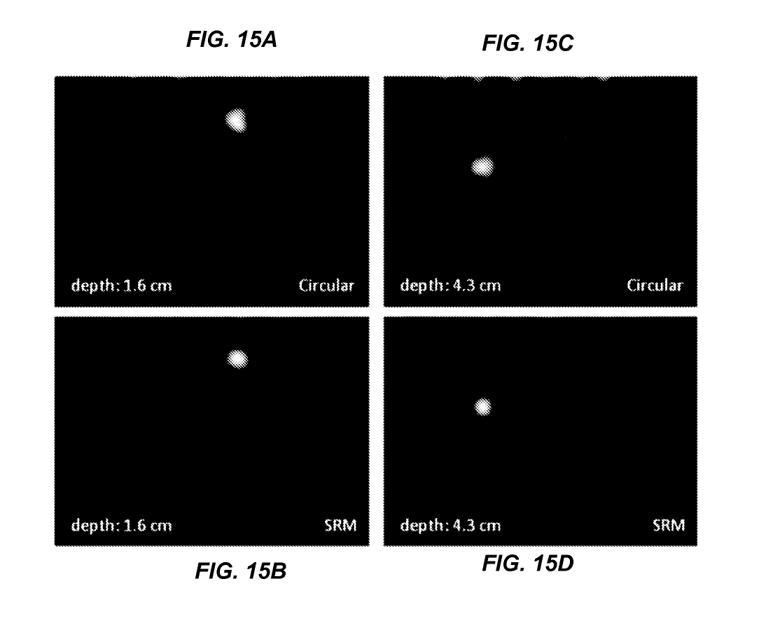

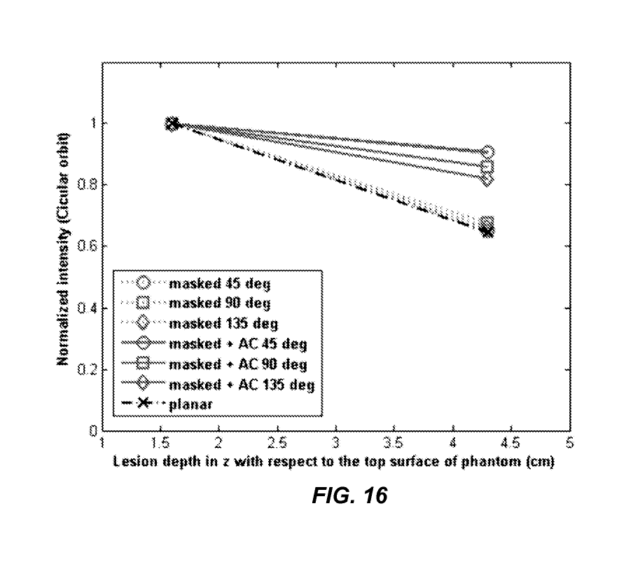

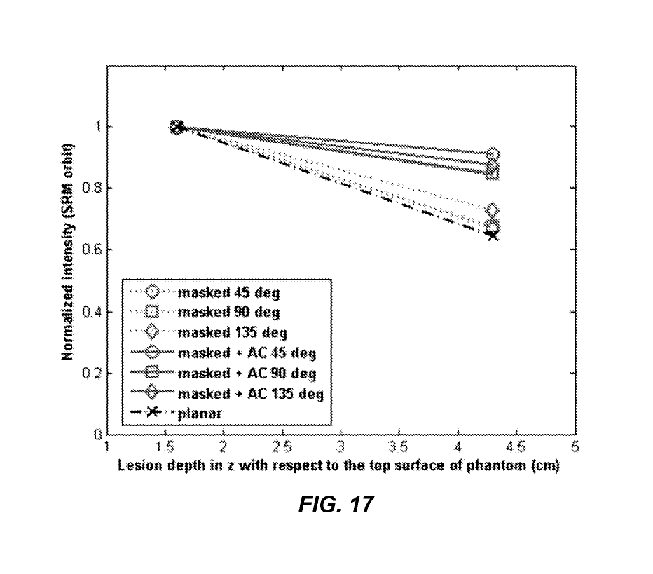

In an example, GEBT reconstruction with 135 degree angular span was performed from circular and SRM orbits of the gelatin breast phantom, wherein regularization and AC were applied in the GEBT reconstruction. As depicted in FIG. 15, the lesion intensity is generally uniform regardless of the orbit applied. Similarly, as depicted in FIGS. 16 and 17, the application of AC according to example increases the intensity of deeper lesions. In an example, the lesion intensity of a lesion at about 4.5 cm depth is 34% less for circular orbits and 31% less for SRM orbits when AC is not applied. In comparison, the lesion intensity is reduced by 14% for circular orbits and 12% for SRM orbits when AC is applied.

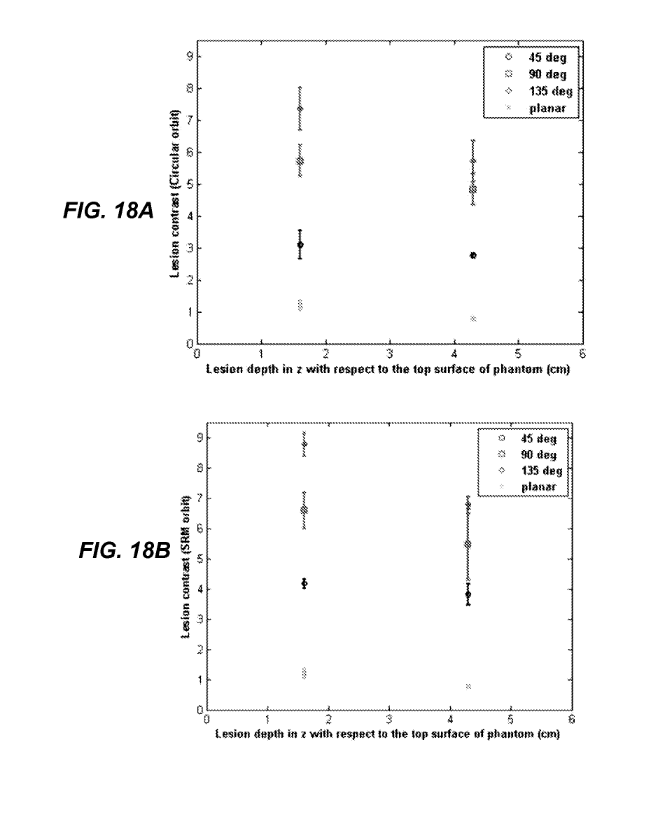

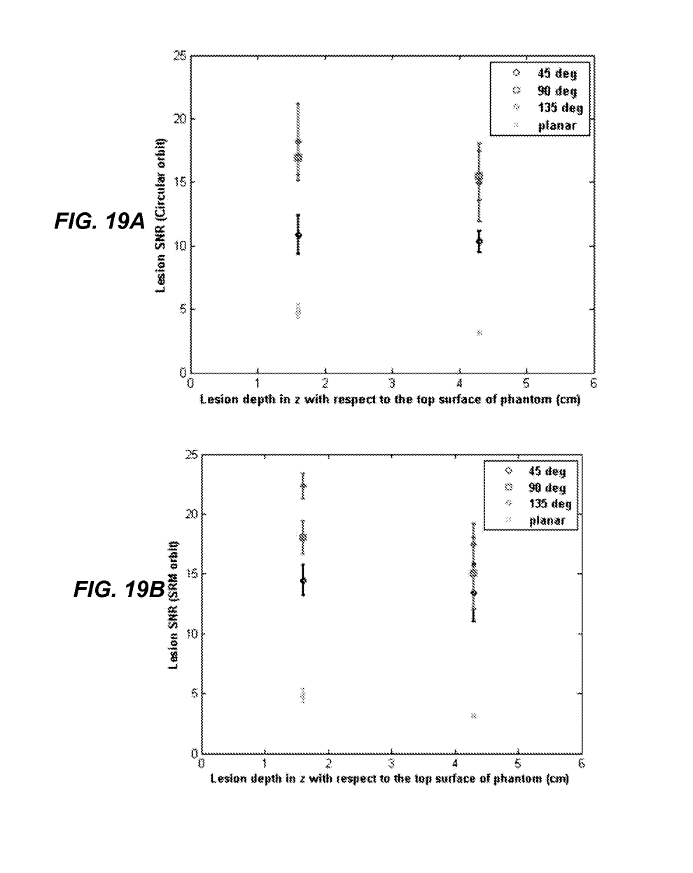

As depicted in FIGS. 18 and 19, as with the box phantom simulation, lesion contrast and SNR are substantially improved when regularization and AC are applied. In an example, depending on the angular range used, the shallow lesion contrast in GEBT is 2.6 to 6.2 times better than in planar scintimammography using a circular orbit and 3.5 to 7.4 times better than in planar scintimammography using an SRM orbit. In at least one example, the lesion SNR in GEBT is also 2.3 to 3.8 times better than in planar scintimammography using a circular orbit and 3.0 to 4.6 times better than in planar scintimammography using an SRM orbit. In at least one example, deep lesion contrast in GEBT is 3.6 to 7.4 times better than in planar scintimammography using a circular orbit and 4.9 to 8.8 times better than that in planar scintimammography using an SRM orbit. In at least one example, the lesion SNR in GEBT is also 3.3 to 5.0 times better than in planar scintimammography using a circular orbit and 4.3 to 5.6 times better than in planar scintimammography using the SRM orbit. For all angular ranges contrast and SNR are higher for a given lesion using the SRM orbit compared to the circular orbit.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method for constructing a three-dimensional image of a breast cancer targeting a radiotracer, comprising:
positioning a gamma-ray camera on a gantry rotatable about an axis of rotation, the gamma-ray camera having a detector surface oriented normal to the axis of rotation;
rotating the gantry to position the gamma-ray camera at a first plurality of angular views within an angular range;
operating the gamma-ray camera to generate at least one two-dimensional gamma-ray image at each angular view; and
reconstructing a single three-dimensional gamma-ray map of radioactivity distribution from the plurality of two-dimensional gamma-ray images, wherein the three-dimensional map comprises a plurality of voxels, wherein voxel values correspond to the tracer concentration at that location.

2. The method of claim 1, further comprising:
defining a region corresponding to biological tissue to be tested; and
excluding voxels outside the region from the reconstruction step.

3. The method of claim 2, further comprising:
positioning an x-ray emitter and the x-ray camera on the gantry rotatable, the x-ray emitter oriented to transmit an x-ray beam through the biological tissue to the x-ray camera;
rotating the gantry to position the x-ray emitter and the x-ray camera at a second plurality of angular views within the angular range;
operating the x-ray emitter and x-ray camera to generate at least one two-dimensional x-ray image at each angular view; and
assembling the plurality of two-dimensional x-ray images into a three-dimensional x-ray image to provide an anatomical frame of reference for interpretation of the gamma-ray map for defining the region of the biological tissue to be evaluated.

4. The method of claim 1, wherein comprising
calculating a volumetric inverse cone structure having dimensions corresponding to a point spread function of the imaging system; and
applying the volumetric inverse cone structure in compiling the two-dimensional gamma-ray images.

5. The method of claim 1, further comprising:
applying an attenuation factor to the constructed radioactivity value of each voxel in the reconstruction step for attenuation correction; and
wherein the attenuation factor is calculated from an attenuation map obtained by at least one of an x-ray transmission system, physical breast measurements, and the known attenuation coefficients of breast tissues.

6. (canceled)

7. The method of claim 1, wherein the gamma-ray camera further comprises a collimator having a plurality of collimator holes and positioned over the detector surface.

8. The method of claim 7, further comprising:
applying a depth-dependent detector blurring factor to the constructed radioactivity value of each voxel in the reconstruction step for resolution recovery, wherein the parameters used for calculating the factor is determined by the dimensions of the collimator holes and the intrinsic spatial resolution of the gamma detector.

9. The method of claim 1, further comprising:
weighting each reconstructed radioactivity value with a depth dependent camera blurring factor.

10. The method of claim 1, further comprising:
summing the weighted radioactivity within the calculated volumetric inverse cone structure as the forward projection.

11-12. (canceled)

13. A system for performing gamma emission tomosynthesis, comprising:
a gantry rotatable about an axis of rotation;
a gamma-ray camera positioned on the gantry and having a surface, the gamma-ray camera oriented such that the surface normal is perpendicular to the axis of rotation, wherein the gantry is rotatable to position the gamma-ray camera at a plurality of angular views within an angular range and the gamma-ray camera is operable to capture at least one two-dimensional gamma-ray image at each angular view; and
a processor for reconstructing a single three-dimensional gamma-ray map of radioactivity distribution from the plurality of two-dimensional gamma-ray images, wherein the three-dimensional map comprises a plurality of voxels, wherein voxel values correspond to the tracer concentration at that location.

14. The system of claim 13, wherein the processor is configured to define a region corresponding to biological tissue to be tested and exclude voxels outside the region from the reconstruction step.

15. The system of claim 13, further comprising:
an x-ray emitter positioned on the gantry and oriented to transmit an x-ray beam through the biological tissue to the x-ray camera; and
an x-ray detector positioned on the gantry opposite the x-ray emitter and oriented to receive the transmitted x-ray beam;
wherein the gantry is rotatable to position the x-ray emitter and the x-ray camera at a second plurality of angular views within the angular range and the x-ray emitter and x-ray camera are operable to capture at least one two-dimensional x-ray image at each angular view;
wherein the processor is configured to combine the plurality of two-dimensional x-ray images into a three-dimensional x-ray image.

16. The system of claim 15, wherein the processor is configured to evaluate the three-dimensional x-ray image in defining the region corresponding to biological tissue.

17. (canceled)

18. The system of claim 13, wherein the processor is configured to calculate the inverse cone structure in three-dimensional reconstruction space apply the inverse cone structure in three-dimensional reconstruction space.

19. The system of claim 13, wherein the processor is configured to apply an attenuation factor to the constructed radioactivity value of each voxel in the reconstruction step for attenuation correction; and
wherein the attenuation factor is calculated from an attenuation map obtained by at least one of an x-ray transmission system, physical breast measurements, and the known attenuation coefficients of breast tissues.

20. (canceled)

21. The system of claim 13, wherein the gamma-ray camera further comprises a collimator having a plurality of collimator holes and positioned over the detector surface.

22. The system of claim 21, wherein the processor is configured to applying a depth-dependent detector blurring factor to the constructed radioactivity value of each voxel in the reconstruction step for resolution recovery, wherein the parameters used for calculating the factor is determined by the dimensions of the collimator holes and the intrinsic spatial resolution of the gamma detector.

23. The system of claim 13, wherein the processor is configured to weight each reconstructed radioactivity value with a depth dependent camera blurring factor.

24. The system of claim 13, wherein the processor is configured to sum the weighted radioactivity within the calculated volumetric inverse cone structure as the forward projection.

25-33. (canceled)

34. A method for constructing a three-dimensional image of a breast cancer targeting a radiotracer, comprising:
positioning a gamma-ray camera, an x-ray emitter and a x-ray camera on a gantry rotatable about an axis of rotation, the gamma-ray camera having a detector surface oriented normal to the axis of rotation, the x-ray emitter oriented to transmit an x-ray beam through the biological tissue to the x-ray camera;
rotating the gantry to position the gamma-ray camera at a first plurality of angular views within an angular range;
operating the gamma-ray camera to generate at least one two-dimensional gamma-ray image at each angular view;
rotating the gantry to position the x-ray emitter and the x-ray camera at a second plurality of angular views within the angular range;
operating the x-ray emitter and x-ray camera to generate at least one two-dimensional x-ray image at each angular view; and
defining a region corresponding to biological tissue to be tested;
excluding voxels outside the region from the reconstruction step;
reconstructing a single three-dimensional gamma-ray map of radioactivity distribution from the plurality of two-dimensional gamma-ray images, wherein the three-dimensional map comprises a plurality of voxels, wherein voxel values correspond to the tracer concentration at that location; and assembling the plurality of two-dimensional x-ray images into a three-dimensional x-ray image to provide an anatomical frame of reference for interpretation of the gamma-ray map for defining the region of the biological tissue to be evaluated.
